(19) United States
(12) Patent Application Publication
(10) Pub. No.: US 2012/0215055 A1
(43) Pub. Date: Aug. 23, 2012

(54) DOUBLE DIAPHRAGM TRANSDUCER

(76) Inventors: Jürgen VAN VLEM, Borgerhout (BE); Bruno Onkelinx, Blanden (BE)

(21) Appl. No.: 13/174,055

(22) Filed: Jun. 30, 2011

Related U.S. Application Data
(60) Provisional application No. 61/444,455, filed on Feb. 18, 2011.

(51) Int. Cl. A61F 11/04

(52) U.S. Cl. 600/25

(57) ABSTRACT

A transducer comprising a main and an auxiliary diaphragm. Each of these diaphragms are coupled to opposite sides of a central shaft located internal to the transducer. Further, the diaphragms may have similar thicknesses and areas. A difference in the pressure internal to the transducer and external to the transducer may result in a force being exerted onto the diaphragm on each side. These forces are transferred from the diaphragms to the central shaft where they may cancel out each other.

### CROSS-REFERENCE TO RELATED APPLICATIONS

This application claims priority to U.S. Provisional Application No. 61/444,455, filed on Feb. 18, 2011. This application is hereby incorporated by reference herein in its entirety.

### BACKGROUND

1. Field of the Invention

The present invention relates generally to transducers, and more particularly, to a transducer having two diaphragms.

2. Related Art

Hearing loss, which may be due to many different causes, is generally of two types, conductive and sensorineural. Sensorineural hearing loss is due to the absence or destruction of the hair cells in the cochlea that transduce sound signals into nerve impulses. Various prosthetic hearing implants have been developed to provide individuals suffering from sensorineural hearing loss with the ability to perceive sound. One such prosthetic hearing implant is the cochlear implant. Cochlear implants use an electrode array implanted in the cochlea of a recipient to bypass the outer and middle ear. More specifically, electrical stimulation is delivered to the inner ear via the electrode array, thereby causing a hearing perception.

Conductive hearing loss occurs when the normal mechanical pathways of the outer and/or middle ear are impeded, for example, by damage to the ossicular chain or ear canal. Individuals suffering from conductive hearing loss typically receive an acoustic hearing aid. Hearing aids rely on principles of air conduction to transmit acoustic signals to the cochlea. Typically, a hearing aid is positioned in the ear canal or on the outer ear to amplify received sound. This amplified sound is delivered to the cochlea resulting in the perception of sound.

Unfortunately, not all individuals suffering from conductive hearing loss are able to derive suitable benefit from hearing aids. For example, some individuals are prone to chronic inflammation or infection of the ear canal. Other individuals have malformed or absent outer ear and/or ear canals resulting from a birth defect, or as a result of medical conditions such as Treacher Collins syndrome or Microtia.

For these and other individuals, another type of hearing prosthesis referred to as a middle ear hearing prosthesis, may be suitable. Middle ear hearing prostheses convert a received sound into a mechanical stimulation. The mechanical stimulation is delivered to the middle or inner ear via an actuator implanted in the middle ear region of the recipient. The mechanical stimulationcauses motion of the cochlear fluid resulting in the perception of the received sound.

### SUMMARY

In one aspect of the present invention, there is provided a transducer, comprising: a housing; a displaceable element located internal to the housing and displaceable relative to the housing; a first diaphragm configured such that a first force generated on the first diaphragm as a result of a pressure difference between a pressure internal to the housing and a pressure external to the housing is directed into the displaceable element; and a second diaphragm configured such that a second force generated on the second diaphragm as a result of the pressure difference between the pressure internal to the housing and the pressure external to the housing is directed into the displaceable element; wherein the first and second diaphragms are configured such that the first force and the second force are directed into the displaceable element in opposite directions.

In another aspect of the present invention, there is provided a method for mechanically stimulating a recipient's ear with a hearing prosthesis having an implantable actuator system comprising an actuator having at least one displaceable element positioned in a hermetically sealed housing, and a first diaphragm and a second diaphragm coupled to opposite sides of the displaceable element, the method comprising: generating an electrical signal based on a received sound; and generating motion of the displaceable element in response to the generated electrical signal.

### BRIEF DESCRIPTION OF THE DRAWINGS

Embodiments of the present invention are described below with reference to the attached drawings, in which:

FIG. 1 is a perspective view of an individual's head in which an auditory prosthesis in accordance with embodiments of the present invention may be implemented;

FIG. 2A is a perspective view of an exemplary DACS, in accordance with embodiments of the present invention;

FIG. 2B is a perspective view of another type of DACS, in accordance with an embodiment of the present invention;

FIG. 3A is a side, cross-sectional view of a prior art actuator system for use in an implantable hearing prosthesis;

FIG. 3B illustrates the actuator system of FIG. 3A where a difference in pressure between Pᵢ and Pₒ causes the diaphragm to deform;

FIG. 4 is a graph illustrating the transfer function of an actuator with respect to varying ambient pressure;

FIG. 5 illustrates an exemplary actuator comprising an auxiliary diaphragm, in accordance with an embodiment of the present invention; and

FIG. 6 illustrates the actuator system of FIG. 5 under a differential pressure in which the material around the center of the diaphragms deflects inward.

### DETAILED DESCRIPTION

Embodiments of the present invention are generally directed to a transducer comprising a main and an auxiliary diaphragm. Each of these diaphragms are coupled to opposite sides of an element (e.g., a central shaft) located internal to the transducer. Further, the diaphragms may have similar thicknesses and areas. A difference in the pressure internal to the transducer and external to the transducer may result in a force being exerted onto the diaphragm on each side. These forces are transferred from the diaphragms to the central shaft where they may counteract each other. The below description will be discussed primarily with reference to one type of transducer, an actuator for use in providing mechanical stimulation to a recipient. However, it should be understood that embodiments of the present invention may also be implemented in other types of transducers, such as transducers configured to convert between electrical and mechanical energy (i.e., from electrical to mechanical or visa versa), such as microphones, actuators, etc.

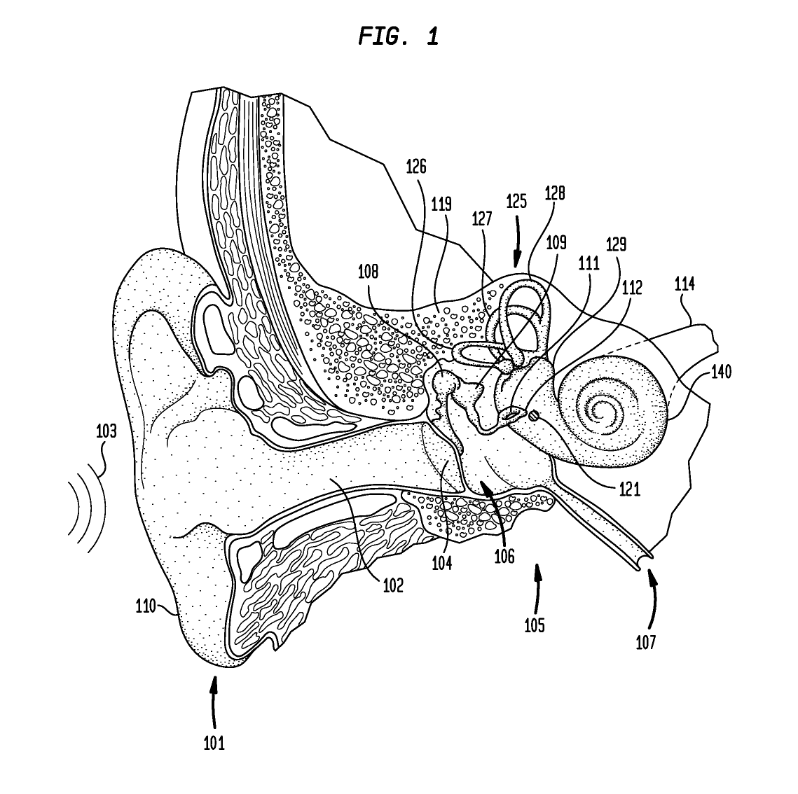

FIG. 1 is a perspective view of an individual's head in which an auditory prosthesis in accordance with embodiments of the present invention may be implemented. As shown in FIG. 1, the individual's hearing system comprises an outer ear 101, a middle ear 105 and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1 are semicircular canals 125. Semicircular canals 125 are three half-circular, interconnected tubes located adjacent cochlea 140. The three canals are the horizontal semicircular canal 126, the posterior semicircular canal 127, and the superior semicircular canal 128. The canals 126, 127 and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

Each canal is filled with a fluid called endolymph and contains a motion sensor with tiny hairs (not shown) whose ends are embedded in a gelatinous structure called the cupula (also not shown). As the skull twists in any direction, the endolymph is forced into different sections of the canals. The hairs detect when the endolymph passes thereby, and a signal is then sent to the brain. Using these hair cells, horizontal canal 126 detects horizontal head movements, while the superior 128 and posterior 127 canals detect vertical head movements.

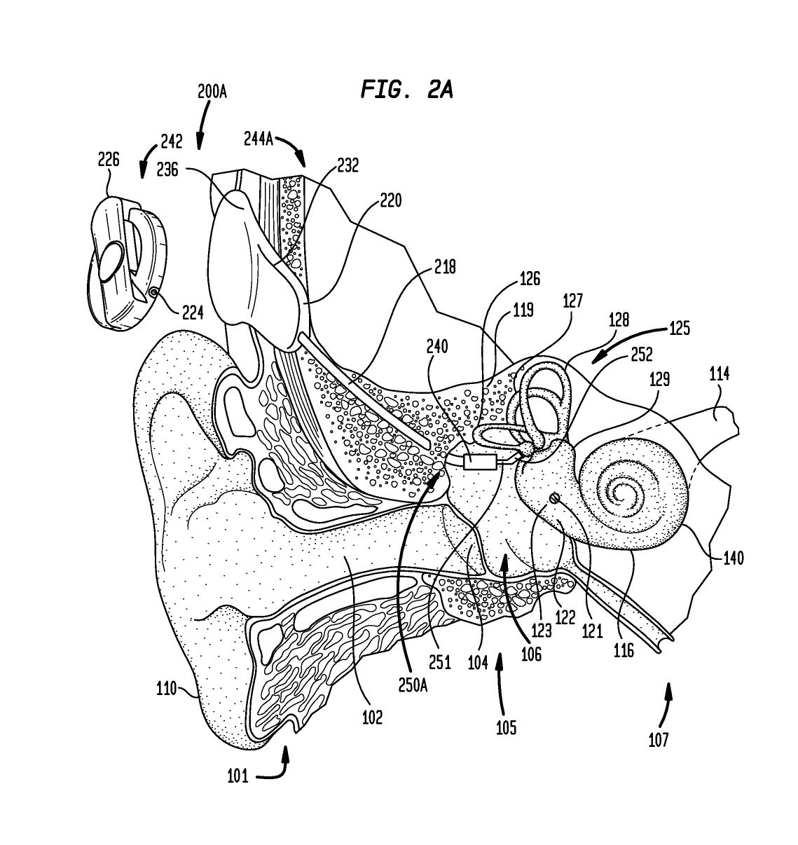

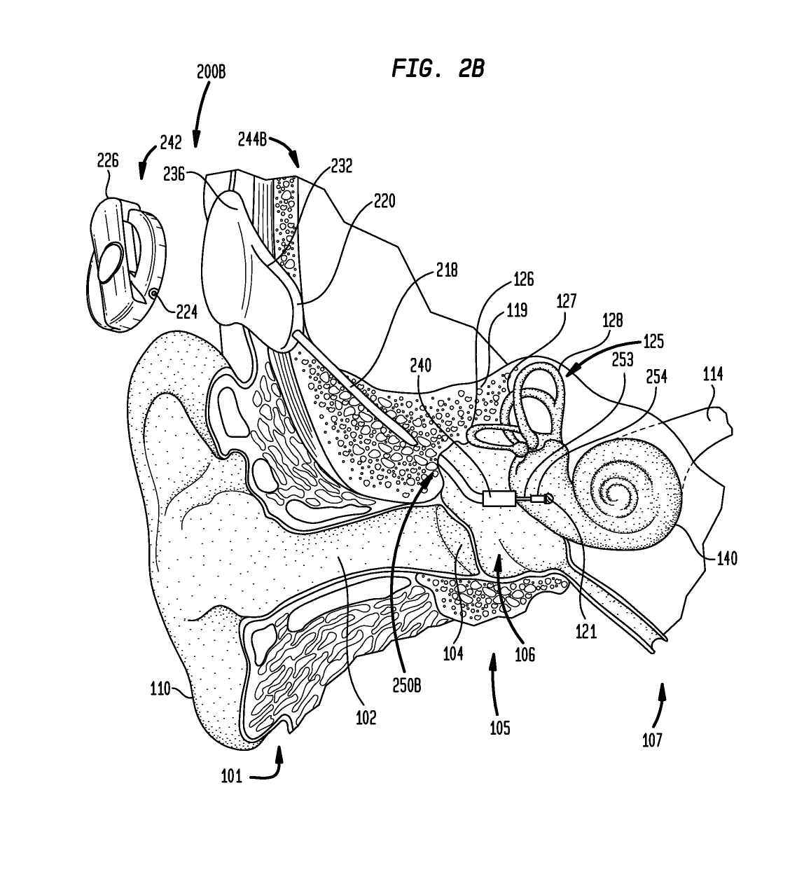

One type of auditory prosthesis that converts sound to mechanical stimulation in treating hearing loss is a direct acoustic cochlear stimulator (DACS) (also sometimes referred to as an “inner ear mechanical stimulation device” or “direct mechanical stimulator”). A DACS generates vibrations that are directly coupled to the inner ear of a recipient and thus bypasses the outer and middle ear of the recipient. FIG. 2A is a perspective view of an exemplary DACS 200A in accordance with embodiments of the present invention.

DACS 200A comprises an external component 242 that is directly or indirectly attached to the body of the recipient, and an internal component 244A that is temporarily or permanently implanted in the recipient. External component 242 typically comprises one or more sound input elements, such as microphones 224 for detecting sound, a sound processing unit 226, a power source (not shown), such as a battery, and an external transmitter unit (also not shown). The external transmitter unit is disposed on the exterior surface of sound processing unit 226 and comprises an external coil (not shown). Sound processing unit 226 processes the output of microphones 224 and generates encoded signals, sometimes referred to herein as encoded data signals, which are provided to the external transmitter unit. For ease of illustration, sound processing unit 226 is shown detached from the recipient.

Internal component 244A comprises an internal receiver unit 232, a stimulator unit 220, and a stimulation arrangement 250A. Internal receiver unit 232 and stimulator unit 220 are hermetically sealed within a biocompatible housing, sometimes collectively referred to herein as a stimulator/receiver unit.

Internal receiver unit 232 comprises an internal coil (not shown), and preferably, a magnet (also not shown) fixed relative to the internal coil. The external coil transmits electrical signals (i.e., power and stimulation data) to the internal coil via a radio frequency (RF) link. The internal coil is typically a wire antenna coil comprised of multiple turns of electrically insulated single-strand or multi-strand platinum or gold wire. The electrical insulation of the internal coil is provided by a flexible silicone molding (not shown). In use, implantable receiver unit 232 may be positioned in a recess of the temporal bone adjacent auricle 110 of the recipient.

In the illustrative embodiment, stimulation arrangement 250A is implanted in middle ear 105. For ease of illustration, ossicles 106 have been omitted from FIG. 2A. However, it should be appreciated that stimulation arrangement 250A may be implanted without disturbing ossicles 106.

Stimulation arrangement 250A comprises an actuator 240, a stapes prosthesis 252 and a coupling element 251. In this embodiment, stimulation arrangement 250A is implanted and/or configured such that a portion of stapes prosthesis 252 abuts an opening in one of the semicircular canals 125. For example, in the illustrative embodiment, stapes prosthesis 252 abuts an opening in horizontal semicircular canal 126. It would be appreciated that in alternative embodiments, stimulation arrangement 250A may be implanted such that stapes prosthesis 252 abuts an opening in posterior semicircular canal 127 or superior semicircular canal 128.

As noted above, a sound signal is received by one or more microphones 224, processed by sound processing unit 226, and transmitted as encoded data signals to internal receiver 232. Based on these received signals, stimulator unit 220 generates drive signals which cause actuation of actuator 240. Stimulator unit 220 may comprise, for example, one or more processors for generation of the drive signals along with a power circuit for providing power to the internal components. The power circuit may comprise, for example, one or more capacitors and/or a battery. Power received by the internal receiver unit 232 may be separated out by the power circuit and stored by the capacitors and/or used to recharge the battery.

This actuation is transferred to stapes prosthesis 252 such that a wave of fluid motion is generated in horizontal semicircular canal 126. Because, vestibule 129 provides fluid communication between the semicircular canals 125 and the median canal, the wave of fluid motion continues into median canal, thereby activating the hair cells of the organ of Corti. Activation of the hair cells causes appropriate nerve impulses to be generated and transferred through the spiral ganglion cells (not shown) and auditory nerve 114 to the brain (also not shown) where they are perceived as sound.

FIG. 2B is a perspective view of another type of DACS 200B in accordance with an embodiment of the present invention. DACS 200B comprises an external component 242 which is directly or indirectly attached to the body of the recipient, and an internal component 244B which is temporarily or permanently implanted in the recipient. As described above with reference to FIG. 2A, external component 242 typically comprises one or more sound input elements, such as microphones 224, a sound processing unit 226, a power source (not shown), and an external transmitter unit (also not shown). Also as described above, internal component 244B comprises an internal receiver unit 232, a stimulator unit 220, and a stimulation arrangement 250B.

In the illustrative embodiment, stimulation arrangement 250B is implanted in middle ear 105. For ease of illustration, ossicles 106 have been omitted from FIG. 2B. However, it should be appreciated that stimulation arrangement 250B may be implanted without disturbing ossicles 106.

Stimulation arrangement 250B comprises an actuator 240, a stapes prosthesis 254 and a coupling element 253 connecting the actuator to the stapes prosthesis. In this embodiment stimulation arrangement 250B is implanted and/or configured such that a portion of stapes prosthesis 254 abuts round window 121.

As noted above, a sound signal is received by one or more microphones 224, processed by sound processing unit 226, and transmitted as encoded data signals to internal receiver 232. Based on these received signals, stimulator unit 220 generates drive signals which cause actuation of actuator 240. This actuation is transferred to stapes prosthesis 254 such that a wave of fluid motion is generated in the perilymph in scala tympani. Such fluid motion, in turn, activates the hair cells of the organ of Corti. Activation of the hair cells causes appropriate nerve impulses to be generated and transferred through the spiral ganglion cells (not shown) and auditory nerve 114 to the brain (also not shown) where they are perceived as sound.

It should be noted that the embodiments of FIGS. 2A and 2B are but two exemplary embodiments of a DACS, and in other embodiments other types of DACS may be implemented. Further, although FIGS. 2A and 2B provide illustrative examples of a DACS system, in embodiments a middle ear mechanical stimulation device may be configured in a similar manner, with the exception that instead of the actuator 240 being coupled to the inner ear of the recipient, the actuator is coupled to the middle ear of the recipient. For example, in an embodiment, the actuator may stimulate the middle ear by direct mechanical coupling via coupling element (e.g., similar to coupling elements 251 or 253) to ossicles 106 (FIG. 1), such to incus 109 (FIG. 1).

An embodiment of the present invention uses an actuator 240 that uses a main and an auxiliary diaphragm. This auxiliary diaphragm may help reduce the actuator's sensitivity to atmospheric pressure changes. Further, this embodiment may provide an actuator whose properties are more constant over time, less susceptible to environmental changes (e.g., ambient pressure) and result in less distortion being perceived by the recipient. The use of an auxiliary diaphragm may also enhance the battery life of the system and the recipient's comfort.

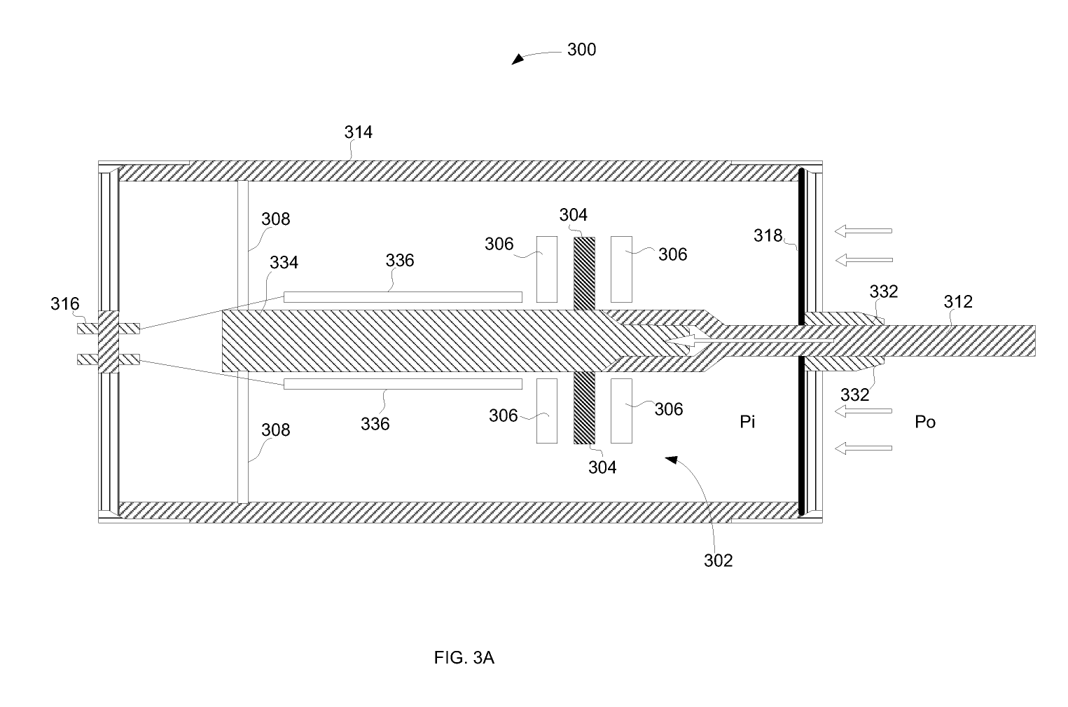

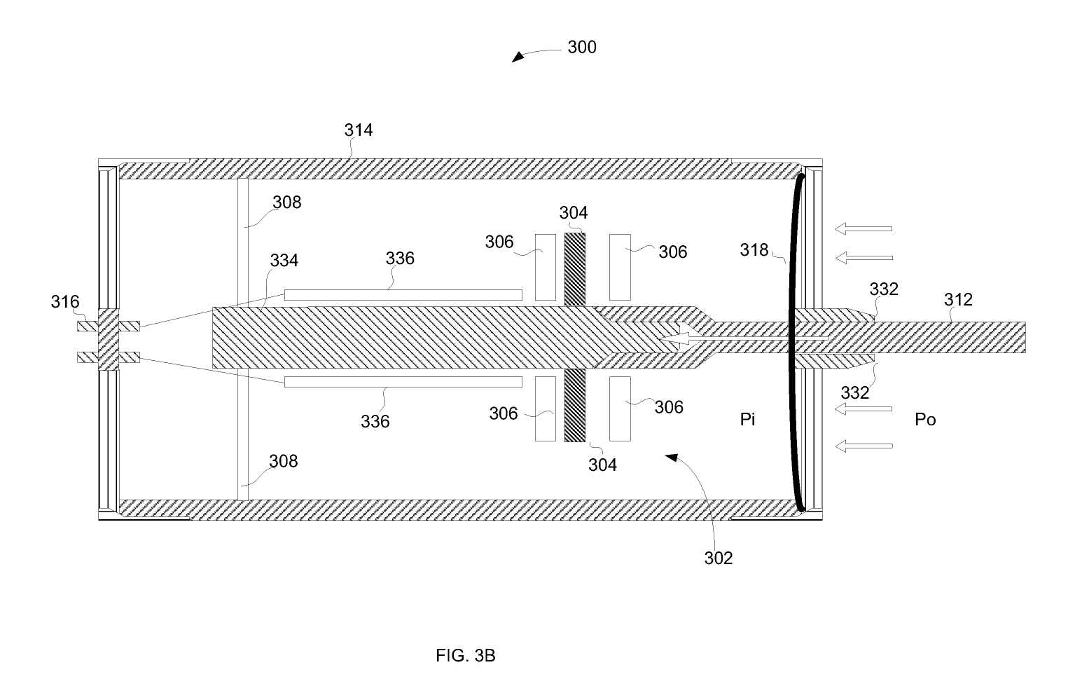

FIG. 3A is a side, cross-sectional view of an actuator system for use in an implantable hearing prosthesis. Actuator system 300 may be used, for example, as actuator 240 of FIG. 1. As shown, actuator system 300 includes an electro-mechanical vibrator 302 including an armature 304, one or more permanent magnets 306, a coil 336, a central shaft 334, and a longitudinal resilient device 308, such as a spring. Actuator system 300 also includes a coupling element 312 connecting vibrator 302 to the recipient's middle or inner ear structure(s), a housing 314, feedthrough 316 and diaphragm 318. Although in the presently discussed embodiment, central shaft 334 is an element having an elongate generally cylindrical shape, in other embodiments the central shaft 334 may be an internal element having a different shape (e.g., rectangular). Housing 314 may filled with a gas or liquid, such as for example, a low viscosity, electrically non-conductive, and non-poisonous liquid such as a biocompatible silicone fluid.

Vibrator 302 operates in accordance with the balanced armature principle. More specifically, vibrator 302 includes a displaceable or moveable element, referred to as armature 304, that is attached to central shaft 334. Armature 304 is configured to move in the magnetic field created by permanent magnets 306. When armature 304 is centered in the magnetic field, there is no net force on the armature, and thus armature 304 is in magnetic equilibrium within the two magnets 306 and is in a “balanced” position.

In operation, drive signals from stimulator unit 220 (FIG. 1) are provided to feedthrough 316 of actuator system 300. The drive signals are then provided to coil 336 to generate a dynamic magnetic field. Central shaft 334 may be manufactured from ferromagnetic material (e.g., iron) such that the magnetic field generated by coil 336 causes movement (e.g., vibrations) of the central shaft 334. Central shaft 334 is coupled to coupling element 312, which transfers the movement to the inner or middle ear of the recipient, such as discussed above with reference to FIGS. 2A-2B. Coupling element 312 may be connected to a connector element 332. Connector element 332 may be further connected to diaphragm 318 thus connecting the coupling element 332 to diaphragm 318. Coupling element 312, connector element 332, and diaphragm 318 may each be manufactured from a biocompatible material, such as titanium.

Changes in static pressure cause a pressure difference between the pressure internal to the actuator system, Pᵢ, and the pressure outside the actuator system, Pₒ. This difference in pressure between Pᵢ and Pₒ may cause diaphragm 318 to deform, thereby changing the stiffness of the diaphragm 318. FIG. 3B illustrates actuator system 300 where a difference in pressure between Pᵢ and Pₒ causes diaphragm 318 to deform. It should be noted that although FIG. 3B illustrates the diaphragm deforming inward, if the difference in pressure is reversed (i.e., Pᵢ>Pₒ) then the diaphragm would deform in the opposite direction (i.e., outward). In the illustrated system, deformation of the diaphragm 318 may change the position of armature 304 between the magnets 306, such that armature 304 moves closer to one of the two magnets 306.

This static bending due to pressure differences preloads the diaphragm giving it an off-center position and a change in its mechanical stiffness. The off-center position also changes the magnetic attraction force and thus the magnetic stiffness. The change in stiffness (magnetic or mechanical) may alter the resonance frequency for the actuator system. These changes in the stiffness (mechanical and/or magnetic) as well a the off-center position may result in a decrease of the efficiency of the actuator.

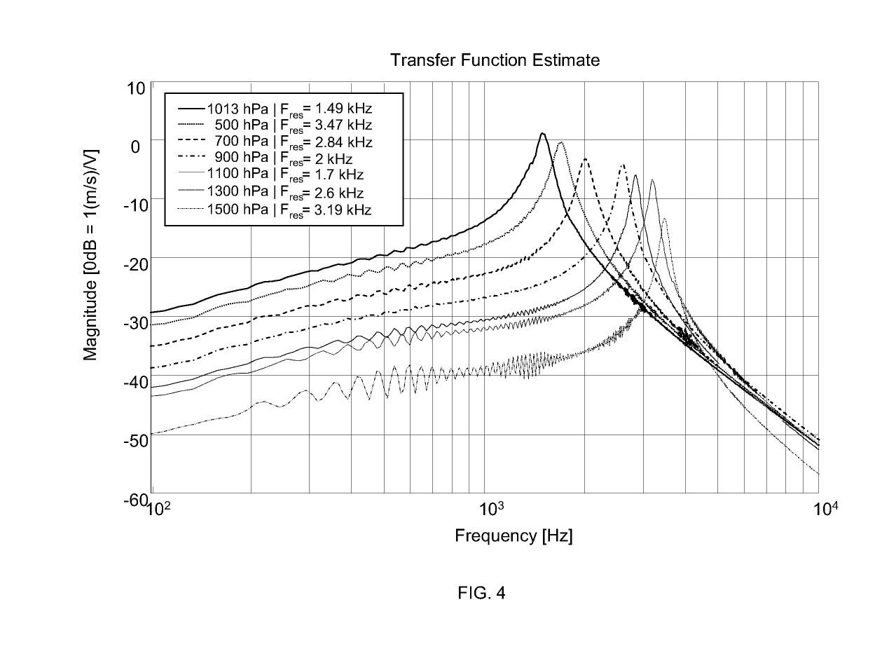

FIG. 4 is a graph illustrating the transfer function of an actuator, such as actuator system 300, having a housing filled with a gas, and the behavior of the system with respect to varying ambient pressure. As illustrated, FIG. 4 plots the transfer functions for an actuator system, such as actuator system 300, for varying ambient pressures (in terms of hectopascals). As shown, actuator system 300 has a resonance frequency (Fᵣₑₛ) of 1.49 kHz at a pressure of 101 hectopascals (hPa). Further, as shown, Fᵣₑₛ=3.47 kHz at 500 hPA, Fᵣₑₛ=2.84 kHz at 700 hPA, Fᵣₑₛ=2 kHz at 900 hPA, Fᵣₑₛ=1.7 KHz at 1100 hPA, Fᵣₑₛ=2.6 kHz at 1300 hPA, and Fᵣₑₛ=3.19 kHz at 1500 hPA.

This shift in resonance frequency may result in the fitting of the sound processor being suboptimal. That is the parameters used for converting received sound to the drive signals may become suboptimal as a result of the changing ambient pressure. This may thus result in a change in the sound perceived by the recipient, which may be annoying or uncomfortable to the recipient. Further, an increase in distortion may result that may be perceived by the recipient. These pressure changes may also result in decreased efficiency of the actuator system, which may reduce the battery life of battery(s) included in the implant system.

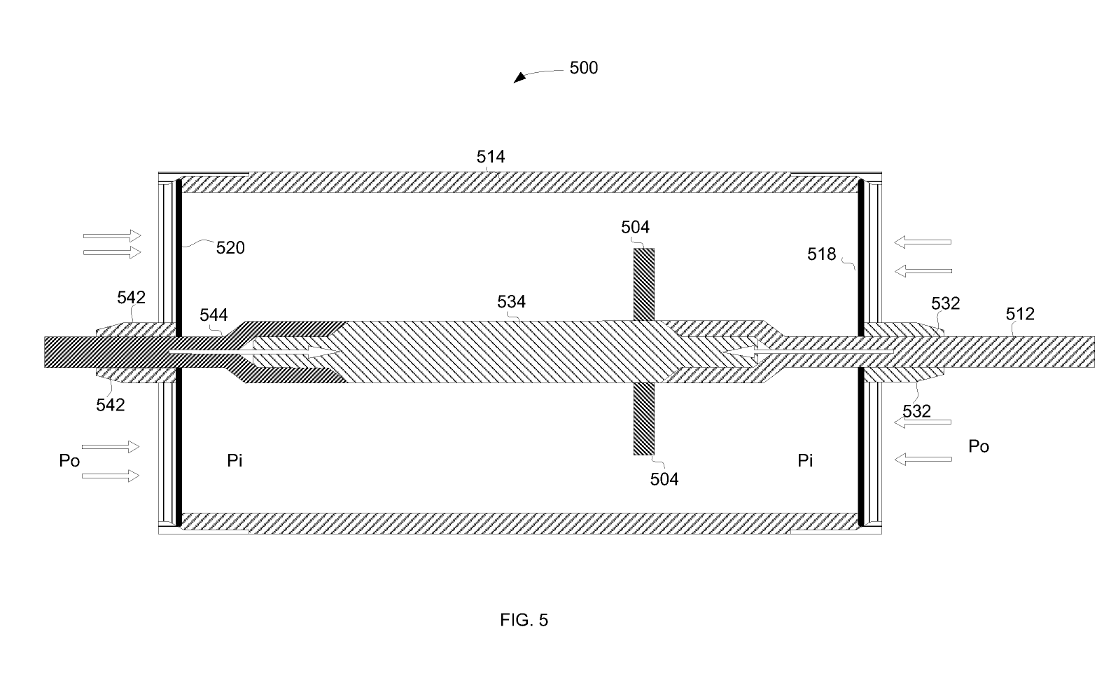

FIG. 5 illustrates an exemplary actuator comprising an auxiliary diaphragm, in accordance with an embodiment of the present invention. For simplification, only the portions of actuator system 500 that will be discussed are illustrated. In actual implementation actuator system 500 may contain additional components, such as those discussed above with regard to FIGS. 3A-3B. For example, actuator system 500 may comprise a feedthrough 316, coil 336, magnets 306, spring 308 etc. such as discussed above with reference to FIGS. 3A-3B. These components may function in a similar manner to as was discussed above with reference to FIGS. 3A-3B. Further, because in the illustrated embodiment, a rod 544, connecting element 542, and diaphragm 520 are located where the feedthrough 316 of FIG. 3 is located, the feedthrough (not shown) in the system of FIG. 5 may be positioned in a different location of actuator system 500. The particular location of the feedthrough may be dependent of the particulars of the implementation of actuator system 500.

Actuator system 500 may be used, for example, as actuator 240 in a system for providing mechanical stimulation to an inner or middle ear of the recipient, such as the systems discussed above with reference to FIGS. 2A-2B.

As illustrated, actuator system 500 comprises a housing 514, a coupling element 512, a first connector element 532, a central shaft 534, an armature 504, a rod 544, a second connector element 542, a main diaphragm 518 and an auxiliary diaphragm 520. Each component (e.g., housing 514, housing 514, a coupling element 512, first connector element 532, rod 544, second connector element 542, main diaphragm 518 and auxiliary diaphragm 520) that may come in contact with bodily fluid when implanted in a recipient may be manufactured from a biocompatible material, such as titanium. Further, the central shaft 534 and/or armature 504 may be manufactured from a ferromagnetic material, such as iron.

As shown, armature 504 may extend perpendicularly from central shaft 534. Further, armature 534 may be positioned such that it is located in the center of a magnetic field, such as a magnetic field generated using magnets (not shown), similar to magnets 306 (FIG. 3). Further, as shown, central shaft 534 is coupled to coupling element 512 and rod 542. Coupling element 512 may be a coupling element such as coupling element 251 (FIG. 2A), coupling element 253 (FIG. 2B), or another coupling element configured for transferring mechanical movement (e.g., vibrations) from the vibrator of the actuator system 500 to the inner or middle ear of the recipient. As illustrated, one end of rod 544 is coupled to central shaft 534 and the other end of rod 544 extends outside the housing 514 such that, when implanted, the end may be exposed to the environment (e.g., body fluids) external to housing 514. It should be noted that, although in the illustrated embodiment rod 544 extends outside housing, in other embodiments rod 544 may not extend outside housing 514. Rather, rod 544 may for example, simply connect diaphragm 520 to central shaft 534 without any portion of rod 544 extending outside housing 514. In such an embodiment, rod 544 may remain entirely within the hermetically sealed enclosure provided by housing 514 and diaphragms 518 and 520. As such, rod 544 may be manufactured from a material other than a biocompatible material.

As shown, coupling element 512 is connected to a connector element 532. Each of these elements 512 and 532 may be manufactured from a biocompatible material such as titanium. Further, elements 512 and 532 may be connected via a seam weld that may, for example, circumferentially extend around the inner diameter of the connector element 532 and outer diameter of the coupling element 512. Although connector element 532 is illustrated as having a nozzle like shape, it should be understood that in other embodiments, connector element 532 may have a different shape.

In addition to being connected to coupling element 512, connector element 532 may also be connected to diaphragm 518. As noted above, diaphragm 518 may be exposed to bodily fluids, as such manufactured from a biocompatible material, such a titanium. In the embodiment of FIG. 5, housing 514 has a cylindrical shape. Further, diaphragm 518 may have a circular shape with a whole in the middle through which coupling element 512 passes. Diaphragm 518 may be connected to connector element 532 via, for example a seam weld. Further, at its outer diameter, diaphragm 518 may be connected to housing 514 (using, for example, a seam weld), such that the housing 514 is hermetically sealed and fluid does not pass into the space within housing 514.

Rod 544, connector element 542, diaphragm 520 and central shaft 534 may be connected in a similar manner. For example, as noted, rod 544 may be connected to central shaft 534. Further, rod 544 is connected to a connector element 542. Each of these elements 544 and 542 may be manufactured from a biocompatible material such as titanium. Further, elements 544 and 542 may be connected via a seam weld that may, for example, circumferentially extend around the inner diameter of the connector element 542 and outer diameter of rod 544. Although connector element 542 is illustrated as having a nozzle like shape, it should be understood that in other embodiments, connector element 542 may have a different shape.

In addition to being connected to rod 544, connector element 542 may also be connected to diaphragm 520. As noted above, diaphragm 520 may be exposed to bodily fluids, as such manufactured from a biocompatible material, such a titanium. In an embodiment of FIG. 5, diaphragm 520 has identical or similar dimensions to diaphragm 518. For example, diaphragm 520 may have a circular shape with a whole in the middle through which rod 544 passes. Further, diaphragm 520 may have the same thickness and be manufactured from the same material as diaphragm 518. Although in the presently discussed embodiments, diaphragms 518 and 520 have a circular shape, it should be understood that in other embodiments, they may have different shapes, such as square or rectangular. Further, as noted, diaphragms 518 and 520 may have matching dimensions and thickness. In an embodiment, diaphragms 518 and 520 may be sufficiently thin to allow diaphragms 518 and 520 to flex during operation of actuator system 500. For example, in an embodiment, diaphragms 518 and 520 may have a diameter equal to or approximately equal to 3.55mm and a thickness equal to or approximately equal to 25 micrometers.

Similar to diaphragm 518, diaphragm 520 may be connected to connector element 542 via, for example a seam weld. Further, at its outer diameter, diaphragm 520 may be connected to housing 514 (using, for example, a seam weld), such that the housing 514 is hermetically sealed and fluid does not pass into the space within housing 514.

In operation, actuator system 500 may function in a similar manner to the above discussed actuator system 300 (FIG. 3A-3B) to provide mechanical stimulation to the recipient for purposes of generating a hearing percept by the recipient. For example, a stimulator unit 220 (FIG. 2A and 2B) may generate drive signals that are provided to feedthrough (not shown) of actuator system 500. The drive signals are then provided to a coil (not shown) to generate a dynamic magnetic field. The dynamic magnetic field generated by the coil (not shown) causes movement (e.g., vibrations) of the central shaft 534. Central shaft 534 is coupled to coupling element 512, which transfers the movement to the inner or middle ear of the recipient, such as discussed above with reference to FIGS. 2A-2B.

Providing the actuator system 500 with an auxiliary diaphragm 520 located opposite to main diaphragm 518 may help cancel out the off-center effect of a pressure differential between the pressure internal, Pᵢ, to actuator system 500 and pressure outside, Pₒ, actuator system 500. For example, as shown, the outside pressure, Pₒ, on the left side of the actuator system 500 exerts a pressure in the right direction on the rod 544 and diaphragm 520 Similarly, the outside pressure, Pₒ, on the right side of the actuator system 500 exerts a pressure in the left direction on the coupling element 512 and diaphragm 518. Because the differential pressure (Pₒ−Pᵢ) on each side will be identical, the pressure on the left side will be equal to the pressure on the right side. In this configuration, the force generated on the left side in the right direction is directed into rod 544, which directs the force into central shaft 534. Similarly, the force generated on the right side in the left direction is directed into coupling element 512, which directs the force into central shaft 534.

Because in this embodiment the diaphragms 518 and 520 have similar or identical dimensions (and accordingly similar or identical areas for integrating the pressure), the force generated on each side will be equal and in opposite directions. Further, in the illustrated embodiment, both diaphragms 518 and 520 are connected to the central shaft 534. Thus, the equal opposite forces will pass into the central shaft 534 where they may cancel out each other.

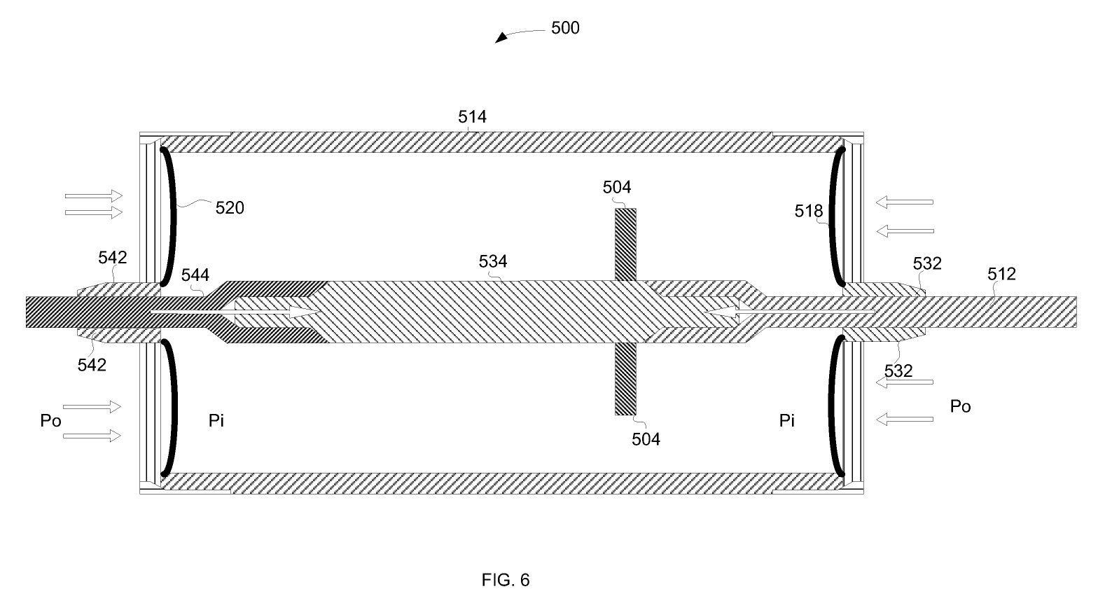

Because in the illustrated embodiment, a differential pressure will result in equal and opposite forces directed into the central shaft, the forces will cancel each other out. Thus, the configuration of actuator system 500, may reduce the likelihood (e.g., prevent) that the armature 504 will move as a result of a difference between the pressure internal and external to the actuator system 500. The efficiency, and thus power consumption, of the actuator may thus be improved over systems in which a differential pressure may result in movement of the armature. Further, because the likelihood is reduced that armature and central shaft move off center as a result of a differential pressure, it is similarly less likely that the diaphragms will offset. This may help reduce the likelihood of changes in the mechanical and/or magnetic stiffness of the diaphragm. It should be noted that although the center of the diaphragm 518 and 520 may not deflect as a result of a differential pressure, the material around the center of the diaphragms 518 and 520 may still bend under pressure. FIG. 6 illustrates actuator system 500 under a differential pressure in which the material around the center of the diaphragms 518 and 520 deflects inward as a result of the pressure outside the actuator system being greater than the internal pressure.

Embodiments of the present invention were described above with reference to an electromagnetic vibrator having two magnets. It would be appreciated that, in alternative embodiments of the present invention, the electromagnetic vibrator may have a single magnet, or more than two magnets. Further, in embodiments, the actuator system may comprise a piezoelectric element in place of the electromagnetic vibrator. Such a piezoelectric element may receive electric drive signals to impart mechanical movement into the central shaft 534, and accordingly the coupling element 512. These mechanical vibrations may be used to generate a hearing percept in the recipient, such as discussed above with reference to FIGS. 2A-2B.

Although the above discussed embodiments, were discussed with reference to one type of transducer, an actuator, in other embodiments the transducer may be a different type of transducer. For example, in an embodiment, the transducer may be a microphone, such as, for example, an implantable microphone configured to be implanted in a recipient. In such an embodiment, the implantable microphone may have a similar configuration to the above discussed system of FIG. 5. However, in such an embodiment, the coupling element may be coupled to a rigid structure (e.g., a bone such as the mastoid bone, a middle ear structure such as one or more bones of the ossicular chain, the ear drum, etc.) such that vibrations (e.g. mechanical movement) in the rigid structure (e.g., bone) are transferred via the coupling element to the central shaft of the transducer. These vibrations may cause movement of the central shaft, which may result in a dynamic magnetic field that is detected by a coil surrounding the central shaft. This magnetic field may result in a current flow through the coil that is then transferred external to the microphone via a feedthrough. The general operation of a microphone is well known to those of skill in the art, and as such is not discussed in further detail herein.

While various embodiments of the present invention have been described above, it should be understood that they have been presented by way of example only, and not limitation. It will be apparent to persons skilled in the relevant art that various changes in form and detail may be made therein without departing from the spirit and scope of the invention. Thus, the breadth and scope of the present invention should not be limited by any of the above-described exemplary embodiments, but should be defined only in accordance with the following claims and their equivalents. All patents and publications discussed herein are incorporated in their entirety by reference thereto.

What is claimed is:

1. A transducer, comprising:
a housing;
a displaceable element located internal to the housing and displaceable relative to the housing;
a first diaphragm configured such that a first force generated on the first diaphragm as a result of a pressure difference between a pressure internal to the housing and a pressure external to the housing is directed into the displaceable element; and a
second diaphragm configured such that a second force generated on the second diaphragm as a result of the pressure difference between the pressure internal to the housing and the pressure external to the housing is directed into the displaceable element;
wherein the first and second diaphragms are configured such that the first force and the second force are directed into the displaceable element in opposite directions.

2. The transducer of claim 1, wherein the transducer is configured for implantation in a recipient.

3. The transducer of claim 1, wherein each of the first and second diaphragms are manufactured from a same material.

4. The transducer of claim 3, wherein the first diaphragm has an area that is equal to an area of the second diaphragm.

5. The transducer of claim 4, where the first diaphragm has a thickness that is equal to a thickness of the second diaphragm.

6. The transducer of claim 1, wherein the housing is a hermetically sealed housing.

7. The transducer of claim 1, further comprising:
a coupling element connected to the displaceable element, wherein the coupling element is configured to couple the transducer to a structure of a recipient.

8. The transducer of claim 7, wherein the structure is a structure of the inner ear or middle ear of the recipient; and
wherein the transducer is configured to transfer mechanical stimulation to the structure via the coupling element in order to cause a hearing percept by a recipient.

9. The transducer of claim 7, wherein the first diaphragm is connected to the coupling element and configured to provide a hermetic seal between the coupling element and a region internal to the housing so as to provide a hermetically sealed housing.

10. The transducer of claim 9, further comprising:
a second element, where a first end of the second element is connected to the displaceable element and a second end extends external to the housing; and
wherein the second diaphragm is coupled to the second element and configured to provide a hermetic seal between the second element and the region internal to the housing.

11. The transducer of claim 10, further comprising
a first connecting element connected to the coupling element and to the first diaphragm so as to couple the coupling element to the first diaphragm; and
a second connecting element connected to the second element and to the second diaphragm so as to couple the second element to the second diaphragm.

12. The transducer of claim 10, wherein each of the coupling element, the housing, the first diaphragm, the second diaphragm, and the second element are manufactured from a biocompatible material.

13. The transducer of claim 12, wherein the biocompatible material is titanium.

14. The transducer of claim 1, wherein the transducer is an actuator system configured to generate motion of the displaceable element in response to an electrical signal.

15. The actuator system of claim 14, wherein the actuator system is an electromechanical actuator comprising one or more magnets.

16. The actuator system of claim 15, wherein the actuator comprises:
a plurality of magnets, and wherein the displaceable element of the actuator comprises an armature positioned between the magnets.

17. The actuator system of claim 15, wherein the actuator is a piezo-electric actuator, and wherein the displaceable element comprises a portion of piezo-electric material.

18. The actuator system of claim 15, wherein the actuator system is a DACS (direct acoustical cochlear system).

19. The transducer of claim 1, wherein the transducer is a microphone configured to sense movement and to generate an electrical signal based thereon.

20. The transducer of claim 19, wherein the microphone comprises a coil configured to detect movement of the displaceable element.

21. The transducer of claim 19, wherein the microphone comprises a piezo-electric material.

22. The transducer of claim 1 wherein each of the first and second diaphragms are manufactured from a bio-compatible material.

23. The transducer of claim 22, wherein the biocompatible material is titanium.

24. The transducer of claim 23, wherein the housing is a titanium housing and wherein each the first and second diaphragms are welded to the housing.

25. A method for mechanically stimulating a recipient's ear with a hearing prosthesis having an implantable actuator system comprising an actuator having at least one displaceable element positioned in a hermetically sealed housing, and a first diaphragm and a second diaphragm coupled to opposite sides of the displaceable element, the method comprising:
generating an electrical signal based on a received sound;
generating motion of the displaceable element in response to the generated electrical signal.

26. The method of claim 25, wherein the actuator comprises a plurality of magnets, and wherein the displaceable element of the actuator comprises an armature positioned between the magnets.

27. The method of claim 26, wherein the actuator is a piezoelectric actuator, and wherein the displaceable element comprises a portion of piezo-electric material.
